(19) United States
(12) Patent Application Publication
(10) Pub. No.: US 2023/0080276 A1
(43) Pub. Date: Mar. 16, 2023

(54) PRESSURISED METERED DOSE INHALERS COMPRISING A BUFFERED PHARMACEUTICAL FORMULATION

(71) Applicant: CHIESI FARMACEUTICI S.p.A., Parma (IT)

(72) Inventor: Enrico ZAMBELLI, Parma (IT)

(21) Appl. No.: 17/801,067

(22) PCT Filed: Feb. 18, 2021
(86) PCT No.: PCT/EP2021/053936

§ 371 Date: Aug. 19, 2022

(30) Foreign Application Priority Data
Feb. 20, 2020 (EP) 20158552.8
Dec. 15, 2020 (EP) 20214098.4

(51) Int. Cl. A61M 15/00; A61K 31/573; A61K 31/137; A61K 31/40; A61K 47/06

(52) U.S. Cl. CPC A61M 15/009; A61K 31/40; A61K 31/137; A61K 31/573; A61K 47/06; A61M 15/0013; A61M 15/0068

(57) ABSTRACT

The present invention generally relates to an aerosol formulation comprising formoterol, beclomethasone dipropionate and glycopyrronium bromide, said formulation being contained in a coated can, particularly useful for the use in a pressurised metered dose inhaler for the treatment of respiratory diseases.

### FIELD OF THE INVENTION

The present invention generally relates to an aerosol formulation comprising at least a LABA, a LAMA, a corticosteroid and a propellant, said formulation being contained in a coated can, particularly useful for the use in a pressurised metered dose inhaler for the respiratory field.

### BACKGROUND OF THE INVENTION

Pressurized metered dose inhalers (pMDIs) are well known devices for administering pharmaceutical products to the respiratory tract by inhalation. A pMDI device typically presents a medical-containing canister (or a “can” as herein referred to), and an actuator housing having a mouthpiece. The can is usually crimped with a metered valve assembly. Depending on the active ingredients and on additional components such as excipients, acids and similar, a final pMDI formulation may be in the form of a solution or a suspension. Solution is generally intended as substantially lacking precipitates or particles, while suspension typically refers to formulation having some undissolved material or precipitates. pMDI devices may use a propellant to expel droplets containing the pharmaceutical products to the respiratory tract as an aerosol. For many years the preferred propellants used in this respect were chlorofluorocarbons derivatives, which are commonly called Freons or CFCs, such as CC13F (Freon 11 or CFC-11), CC12F2 (Freon 12 or CFC-12), and CClF2-CClF2 (Freon 114 or CFC-114). Due to international concern that fully and partially halogenated chlorofluorocarbons possess a critical value of Global Warming Potential (GWP) impacting the earth’s protective ozone layer, many countries entered into an agreement, the Montreal Protocol, stipulating that their manufacture and use should be severely restricted and eventually phased out completely. Consequently, hydro fluoroalkanes (HFAs), in particular 1,1,1,2-tetrafluoroethane (HFA 134a) and 1,1,1,2,3,3,3-heptafluoropropane (HFA 227a) have been identified and accepted as substitutes to the CFCs in the pharmaceutical sector. Since then, the hydro fluoroalkanes propellants HFA 134a and HFA 227a have been widely used in the respiratory field, particularly considering their efficacy and compatibility with many active ingredients such as corticosteroids, LABA or antimuscarinic drugs.

However, despite the efficacy of said HFA propellants and despite their wide application in many pharmaceutical drugs already on the market, the possibility to have an alternative class of propellant and alternative means for obtaining effective pMDI devices are always under consideration. As general reference in this sense, see e.g. “Pharmaceutical Inhalation Aerosol Technology”, Third Edition 2019, Anthony J. Hickey et Al. wherein at page 440, Table 18.3 several propellants potentially suitable for medical use have been compared in terms of Global Warning Potential.

This is related for instance to the optimization of the mechanical components of the pMDI device, such as the valves or the cans, or even the possibility to have propellant-free nebulization devices, spray drying systems, or devices characterized by a more environmental friendly impact.

An additional feature worth to be considered when discussing a pMDI device, is the apparent pH and the water content of the formulation nebulized by said device. As a general reference in this sense, see e.g. WO 01/89480 and WO 03/074024.

Fluorocarbon polymers are commonly used to coat the interior can surfaces of pMDIs to eliminate particle adhesion, or deposition on can walls, i.e. avoiding the sticking, for suspension formulations and to avoid the formation of sub-products.

EP0820323 describes a pMDI having part or all of its internal surfaces coated with one or more fluorocarbon polymers for dispensing an inhalation drug formulation comprising salmeterol, and a fluorocarbon propellant, optionally in combination with one or more other pharmacologically active agents, wherein the coating of the interior can surfaces significantly reduces or essentially eliminates the problem of adhesion or deposition of salmeterol.

WO 2015/101576 describes a pMDI device particularly suitable for the use with a formoterol, beclomethasone dipropionate and glycopyrronium bromide solution, contained in a FEP coated can. As therein disclosed, the formulation contained in a FEP coated can is endowed with an improved stability and reduced amount of degradation products, mainly with regards to the N-(3-bromo)-[2-hydroxy-5-[1-hydroxy-2-[1-(4-methoxyphenyl)propan-2-ylamino]ethyl] phenyl]formamide. This product (identified as DP3) is, in fact, a particular degradation product originated by the interaction of formoterol and bromine ions from glycopyrronium bromide when the two active ingredients are dissolved in a HFA ethanol system in the presence of an acid, particularly hydrochloric acid.

EP2706987 describes a formulation for use in a pMDI device comprising beclomethasone dipropionate and HFA152, particularly suitable for the treatment of respiratory diseases.

WO2018/051131 describes in Example 1, Table 4 a pharmaceutical formulation comprising beclomethasone dipropionate and formoterol fumarate dihydrate, a propellant comprising 1,1-difluoroethane (HFA 152a), optionally a LAMA agent such as glycopyrrolate bromide and glycerol. However, WO2018/051131 does not discloses a coated can suitable for use with the above formulation.

WO2018/051130 describes a pharmaceutical formulation comprising a drug component comprising at least one pharmaceutically acceptable salt of glycopyrrolate and a propellant component comprising HFA 152a, wherein said formulation exhibits satisfactory stability without the use of acid stabilizers.

WO2019236559, published Dec. 12, 2019, describes pharmaceutical compositions for use in a pMDI device comprising beclomethasone dipropionate, formoterol fumarate dihydrate, glycopyrronium, a propellant selected from HFA 134a, 227a and 152a, co-solvent, an organic acid(s) and optionally water.

US20160324778, describes medicinal composition for use in a pressurized medicinal composition comprising a propellant selected form HFO-1234yf (2,3,3,3-tetrafluoropropene) and HFO-1234ze (1,3,3,3-tetrafluoropropene) and one or more active ingredient such as formoterol and beclomethasone dipropionate, wherein the active ingredient is in the form of a suspension or a solution with the propellant.

Although the above mentioned prior art provides effective formulations and devices technical arrangements, there is still the need to find a proper pMDI device for use in the respiratory field for the treatment of e.g. asthma and/or COPD, which not only contemplates the reduction of the greenhouse warming potential (GWP), but that also conveniently provides a good stabilization system, particularly regarding the calibration and maintenance of the apparent pH of the formulation contained in said device. It is in fact noticed that the prior art is silent about a proper and practical way to buffer the apparent pH of a formulation suitable for a pMDI device, comprising at least a corticosteroid, a LABA agent, a LAMA agent and a propellant. The apparent pH is in fact a crucial parameter which can impact many aspects of a pMDI formulation, especially when in the form of a solution, such as for instance, stability of the LABA and/or LAMA agents, shelf life, consistent delivery of medication in aerosol from the MDI, the reproducibility of the final formulation and the maintenance of optimal chemical conditions within the can.

We have unexpectedly found that it is possible to stabilize the apparent pH of a formulation suitable for pMDI device comprising at least a corticosteroid, a LABA, a LAMA and a proper HFA or HFO propellant, by means of an internally coated can provided with a dedicated metering valve system.

We have surprisingly found that the use of an internally coated can provided with a dedicated metering valve system avoids the presence of a buffering agent to maintain stable the apparent pH of a pMDI formulation. In fact, the internally coated can according to the invention is able to stabilize the apparent pH, even for a prolonged period, as demonstrated in the herein below experimental part. In this sense, the coated can of the invention is able to act as an apparent pH buffering system and the use of a dedicated metering valve further increases the apparent pH buffering action of the coated can.

Advantageously, said coated can provided with a proper valve system containing at least a corticosteroid, a LABA, a LAMA and the selected HFA or HFO propellant of the invention are readily used in a pMDI device for the treatment of respiratory diseases, such as asthma and/or COPD, also guaranteeing a good stability of the chemical components over the time, excellent aerosolizing performance, along with a low GWP.

### SUMMARY OF THE INVENTION

In one aspect, the present invention refers to a can for use in a pMDI device, said can containing a formulation comprising at least a corticosteroid, a LABA agent, a LAMA agent and a HFA 152a or HFO propellant, being said can internally coated by a coating comprising at least a com-pound selected from: an epoxy-phenol resin, a perfluorinated polymer, a per-fluoroalkoxyalkane polymer, a perfluoroalkoxyalkylene polymer, a per-fluoroal-kylene polymer, poly-tetrafluoroethylene polymer (Teflon), fluorinated-ethylene-propylene polymer (FEP), polyether sulfone polymer (PES), a fluorinated-ethylene-propylene polyether sulfone polymer (FEP-PES), a polyamide, polyi-mide, polyamideimide, polyphenylene sulfide, plasma, mixtures or combinations thereof, wherein said can is provided with a valve having at least one gasket made of a material comprising at least one polymer selected from low-density poly-ethylene, butyl such as chlorobutyl or bromobutyl, butadiene-acrylonitrile, neo-prene, EPDM (a polymer of ethylenepropylenediene monomer), TPE (thermo-plastic elastomer), cycloolefin copolymer (COC) or combination thereof. In one additional aspect, the present invention refers to the above indicated coated can, wherein said formulation comprising at least a corticosteroid, a LABA, a LAMA agent and HFA or HFO propellant is a solution, preferably also comprising a mineral or organic acid and/or a co-solvent.

In a further aspect, the invention refers to a pMDI device for use in the respiratory filed, particularly for treatment of asthma and/or COPD, comprising the above indicated coated can.

### DETAILED DESCRIPTION OF THE INVENTION

Unless otherwise defined, all technical and scientific terms used herein have the same meanings as commonly understood by the skilled in the art.

The “molar ratio” between formoterol or a salt thereof or a solvate of said salt and the acid is calculated considering the number of moles of formoterol or a salt thereof or a solvate of said salt within the formulation and number of moles of the selected acid in the formulation.

Unless otherwise provided, the term “formoterol fumarate” or “FF” refers to (R,R)-(±)formoterol fumarate or dihydrate thereof.

Unless otherwise indicated the term “LABA” or “LABA agent” includes in its meaning a long acting beta 2 agonist, as known in the art.

Unless otherwise indicated the term “LAMA” or “LAMA agent” includes in its meaning a long acting muscarinic receptor antagonist, as known in the art.

The term “% w/w” means the weight percentage of the component in respect to the total weight of the formulation.

The term “% w/v” means the weight percentage of the component in respect to the total volume of the formulation.

A “stable” composition as defined herein means that the content of residual active ingredient is of at least about 90% w/w (which is the content percent by weight with respect to its initial content at time 0), preferably of at least about 95% w/w, and that the total content of degradation product is of not more than about 10% by weight with respect to initial content of the active ingredient at time 0, preferably of not more than about 5% by weight, at a given time point, as measured by HPLC/UV-VIS.

Regarding the term “apparent pH” as herein intended, it is noticed that the calculation of the pH is generally characteristic of aqueous liquid, e.g. where water is the dominant component. In relatively aprotic solvents such as the HFA system of the present invention, protons are non-hydrated and their activity coefficients can differ from those in aqueous solution. Although the Nerst equation (describing potential of electrochemical cell as a function of concentrations of ions taking part in the reaction) with respect to electromagnetic field (EMF) applies and the pH-meter glass electrode system will generate a variable milli-volt output according to proton concentration and vehicle polarity, the pH meter reading represents the “apparent pH” according to the present invention. In this direction, the apparent pH according to the invention can be measured by technologies known in the art, as e.g. indicated in “Correlation between Apparent pH and Acid or Base Concentration in ASTM Medium” Orest Popovych, Analytical Chemistry 1964, 36,4,878-882; Analytical Standard Test Method (ASTM) D6423 - 19 “Standard Test Method for Determination of pH of Denatured Fuel Ethanol and Ethanol Fuel Blends”.

As above mentioned, the present invention unexpectedly shows that when a coated can provided with a dedicated valve system as herein described in details, suitable for a pMDI device, is used to contain a proper formulation comprising at least a corticosteroid, a LABA agent, a LAMA agent and a HFA or HFO propellant, the apparent pH of such formulation can be conveniently buffered between about 2.5 and 5, preferably between about 3 and 4.5, depending e.g. on the components of the formulation and/or on their amounts, as herein below described. Having such a buffering system brings several advantages, such as the increase in the stability of the formulation over the time, particularly regarding the formoterol amount, good shelf life, the reproducibility of the final formulation, the maintenance of optimal chemical conditions within the can and consistent delivery of medication in aerosol from the MDI.

In particular, having a stable apparent pH by means of an internally coated can provided with a dedicated valve system avoids the addition of an external traditional acid-base buffering system, that would lead to a more complex formulation; the combined use of a coated can together with a dedicated metered valve further increase the stability of the formulation acting as apparent pH buffering system. On the contrary, non-internally coated cans do not show the effect of keeping the apparent pH constant over time for a pMDI solution formulation, as demonstrated in the herein below comparative examples.

Thus, in one embodiment, the invention refers to a can provided with a dedicated valve system for use in a pMDI device, containing a formulation as herein described and claimed, characterized by the fact that the apparent pH of said formulation is stabilized at a value between about 2.5 and 5, preferably between about 3 and 4.5. In other words, the invention also refers to the herein described and claimed coated can, suitable for buffering the apparent pH of a formulation comprising at least a corticosteroid, a LABA, a LAMA and a HFA or HFO propellant, between about 2.5 and 5, preferably between about 3 and 4.5.

The apparent pH of the pMDI formulation is influenced by the composition of the formulation, e.g. with reference to the concentration of the acid and the like, and the setting of a proper value may be achieved by selecting a proper amount and type of LABA, LAMA and/or corticosteroid agent, or by adding additional components to the formulation, as herein below described.

As far as the can is concerned, a coated can known in the art may be suitably used in the present invention. Thus, the can may be made of a metal, e.g. aluminum, or metal alloys, stainless steel or anodized aluminum, fluorine passivated aluminum and the like. Alternatively, the can may be made of plastic or any other suitable material. Preferably the can is made of aluminum, optionally anodized, or stainless steel, properly coated. The coating is typically applied to the internal surface of the can, thus providing an internal layer acting as interface between the internal surface of the can, and the formulation therein contained. By that, the internal coating will prevent the adherence of a component of the formulation on the can surface, also setting a pH buffering system. Typically, the internal coating will form a coating layer characterized by having a thickness that meets the uniformity and homogeneity requirements, as tested using e.g. WACO enamel rater instrument as e.g. available on the market. The internal coating will cover at least 50% of the internal surface of the can, preferably at least 95%, even more preferably, at least 99%.

In this regards, a suitable coated can of the invention may have part or all of its internal surfaces coated with an inert organic or inorganic coating preferably comprising: an epoxy-phenol resin, a perfluorinated polymer, a perfluoroalkoxyalkane polymer, a perfluoroalkoxyalkylene polymer (PFA), a perfluoroalkylene polymer, poly-tetrafluoroethylene polymer (PTFE or Teflon), fluorinated-ethylene-propylene polymer (FEP), polyether sulfone polymer (PES), a fluorinated-ethylene-propylene polyether sulfone polymer (FEP-PES), a polyamide, polyimide, polyamideimide, polyphenylene sulfide, plasma, mixtures or combinations thereof.

By way of example, the term “FEP-coated” refers to a coating layer comprising FEP, and optionally additional components including additives, adhesives, aggregation agents such as PES, isobutylketone and the like.

The above listed polymers may be used in combination with additional components, or as part of a polymeric mixture, obtained e.g. by blending together two or more polymeric compounds. In this direction, the internal coating of the can according to the invention is intended to comprise also said mixtures or combinations. In one embodiment, the coated can of the invention is a FEP or a PTFE coated can, or more preferably a FEP-PES coated can. In the case of FEP-PES coated, the PES acts as an intermediate layer between the internal surface and the FEP polymer, thus assuring an even more uniform and homogenous coating. It has in fact to be noted that, when suitable, more than one coating may be applied to the internal surface of the can, thus forming a bilayer or a multilayer coating having improved homogeneity and stability.

In one embodiment of the invention, the can is an aluminum can, characterized by having an internal coating comprising a FEP-PES polymer. Suitable aluminum FEP coated cans for the invention are those e.g. commercially available and used in the field.

As demonstrated in the herein below experimental part, when a formulation in form of a solution comprising beclomethasone dipropionate (BDP), formoterol fumarate dihydrate and, glycopyrronium bromide and HFA152a propellant is contained in a FEP coated can provided with dedicated valve system according to the invention, the apparent pH of said formulation is conveniently maintained at a selected value, even for prolonged period of time.

In one embodiment, the corticosteroid component of the formulation contained in the coated can provided with a dedicated valve system according to the invention, is selected from the group consisting of: budesonide, beclomethasone (BDP), e.g. as the mono or the dipropionate ester, flunisolide, fluticasone, e.g. as the propionate or furoate ester, ciclesonide, mometasone, e.g. as the furoate ester, mometasone desonide, rofleponide, hydrocortisone, prednisone, prednisolone, methyl prednisolone, naflocort, deflazacort, halopredone acetate, fluocinolone acetonide, fluocinonide, clocortolone, tipredane, pred-nicarbate, alclometasone dipropionate, halometasone, rimexolone, deprodone propionate, triamcinolone, betamethasone, fludrocoritisone, desoxycorticosterone, rofleponide, eti-prednol dicloacetate, wherein, beclomethasone dipropionate (BDP) and budesonide are particularly preferred. In a still preferred embodiment, the corticosteroid component is beclomethasone dipropionate (BDP).

According to another embodiment, the amount of the corticosteroid component according to the present invention is comprised between 0.01-0.7 % w/w, more preferably between 0.05-0.5 % w/w, even more preferably between 0.1-0.3 % w/w.

As far as the LABA component of the formulation contained in the coated can according to the invention is concerned, this is preferably selected from the group consisting of: fenoterol, formoterol fumarate, formoterol fumarate dihydrate, arformoterol, carmoterol (TA-2005), indacaterol, milveterol, bambuterol, clenbuterol, vilanterol, olodaterol, abediterol, terbultaline, salmeterol, diastereoisomeric mixtures, and a pharmaceutically acceptable salt thereof or hydrate thereof. In one embodiment, the LABA is formoterol fumarate, preferably formoterol fumarate dihydrate.

Alternatively, the formulation of the present invention may comprise salbutamol, (R)-salbutamol (levalbuterol) and a pharmaceutically acceptable salt thereof or hydrate thereof.

Preferably, the amount of LABA according to the present invention is comprised between 0.0005-0.04 % w/w , more preferably between 0.001-0.03% w/w , even more preferably between 0.005-0.02 % w/w .

In one embodiment, the LAMA agent component of the formulation contained in the coated can according to the invention, is selected from the group consisting of: glycopyrronium, methscopolamine, ipratropium, oxitropium, trospium, tiotropium, aclidinium and umeclidinium or pharmaceutically acceptable salts. In one preferred embodiment, the LAMA agent is glycopyrronium bromide. Preferably, the amount of LAMA according to the present invention is comprised between 0.001 to 0.08% (w/w) , preferably from 0.005 to 0.06% (w/w) , more preferably from 0.01 to 0.04% (w/w) .

The propellant of the formulation contained in the coated can according to the invention is selected from HFA 152a and hydrofluoroolefins (HFOs).

In one embodiment, the HFO propellant of the formulation contained in the coated can according to the invention is selected from the group consisting of:1,3,3,3-tetrafluoropropene (HFO-1234ze) and 2,3,3,3-tetrafluoropropene (HFO-1234yf). Preferably the propellant is HFO-1234ze.

In one preferred embodiment the propellant is HFA152a.

The formulation contained in a coated can according to the invention may be in the form of a suspension or a solution. In one embodiment, the selected corticosteroids, LABA and LAMA components are preferably dissolved in the HFA or HFO propellant as above defined, thus providing a solution. Hence, in one particularly preferred embodiment, the invention refers to a FEP coated can for use in a pMDI device, said FEP coated can containing a solution comprising at least beclomethasone dipropionate, formoterol fumarate dihydrate, glycopyrronium bromide and HFA 152a.

As above set forth, in one embodiment the formulation contained in a coated can according to the invention, may optionally further comprise additional components such as excipients, additives, solvents, co-solvents, acids, low volatility components or even active ingredients. The addition of said components may be suitably calibrated in order to module e.g. the chemical-physical properties of the formulation and/or to set a proper apparent pH which is desired to be kept constant, according to the present invention. In this respect, in one preferred embodiment, the invention refers to a coated can for use in a pMDI device as above described, said coated can containing a formulation comprising a corticosteroid, a LABA agent, a LAMA agent, an HFA or HFO propellant, and optionally a co-solvent and/or an acid and/or a low volatile component.

Preferably, said co-solvent is a polar compound able to increase the solubility of the components within the formulation. Examples of suitable co-solvents are aliphatic alcohols having from 1 to 4 carbon atoms, such as methanol, ethanol, propanol, isopropanol and the like, preferably ethanol, more preferably anhydrous ethanol.

When present, said co-solvent is used in an amount comprised between 5%w/w and 20%w/w , more preferably between 10% and 15%.

In one embodiment, the acid may be a mineral or organic acid, preferably selected from: hydrochloric, hydrobromic acid, nitric acid, fumaric acid, phosphoric acid and citric acid, maleic acid, acetic acid, xinafoic acid, oxalic acid, lactic acid, 2-methyl propionic acid, malic acid, butanoic acid, tartaric acid, propionic acid, pentanoic acid, succinic acid, glycolic acid, hexanoic acid, malonic acid, glutaric acid, formic acid, adipic acid, ascorbic acid, benzoic acid, glucuronic acid or mixtures thereof, being hydrochloric particularly preferred. According to a still preferred embodiment, the acid is hydrochloric acid, concentrated or diluted, preferably 1 M. Preferably, when the acid is HCl 1 M it is used in an amount comprised between 0.001-0.08%w/w , preferably between 0.005-0.06%, more preferably between 0.01-0.04%.

In general, the amount of the chosen acid is preferably selected in order to have a final apparent pH of the solution comprised between about 2.5 and 5, preferably between 3 and 4.5, as above set forth. According to the invention, by using a coated can provided with a dedicated valve system, the selected apparent pH is maintained stable and substantially unvaried over the time, even when said pH is set by the presence of an acid, thus solving the problem of how to control and stabilize the apparent pH of a formulation suitable for pMDI application, comprising at least a corticosteroid, a LABA agent, a LAMA agent and a propellant, in the presence of an inorganic or organic acid.

In a still preferred embodiment, the pMDI solution of the invention consist of a LABA, a LAMA a corticosteroid dissolved in a system comprising or consisting of HFA152a, HCl 1 M and EtOH. According to this still preferred embodiment, the LABA, LAMA and corticosteroid are, respectively formoterol fumarate dihydrate glycopyrronium bromide, and beclomethasone dipropionate.

As it is will be recognized, also these last described embodiments are to be intended as included in the scope of the present invention, also in any possible combination with all the other preferred embodiments, as herein above and below set forth.

In one embodiment of the invention, the molar ratio between the LABA and the acid, when present, is comprised between 0.50 to 1.50, preferably between 0.9 and 1.1. It is in fact noticed that in this range the stability of the final formulation is increased up to a particularly convenient degree.

When present, the low volatility component has a vapor pressure at 25° C. lower than 0.1 kPa, preferably lower than 0.05 kPa, preferably selected from the group consisting of: glycols, propylene glycol, polyethylene glycol, glycerol or esters thereof, ascorbyl palmitate, isopropyl myristate and the like, wherein isopropyl myristate and glycerol are particularly preferred.

According to one embodiment, the formulation of the present invention contains an amount of water preferably below 3000 ppm, more preferably below 2000 ppm, still more preferably below 1500 ppm on the total weight of the formulation.

It is worth to note that by the present invention, the problem of how to effectively buffer an apparent pH of a pMDI formulation for commercial purposes comprising a corticosteroid, a LABA agent, a LAMA agent and a HFA or HFO propellant is surprisingly solved in the absence of additional buffering ingredients or agents, which could nevertheless compromise the stability and/or the efficacy of the formulation contained in the can. Also from a manufacturing point of view, the present invention allows the preparation of a pMDI device ready for use, comprising a coated can as herein detailed, with a simple and consolidated manufacturing process. Even further, the use of a green propellant such as HFA 152a allows the present invention not only to solve the above expressed problems, but also to address potential environmental concerns arising from a prolonged use of other fluorinated propellants.

As above indicated, the coated can for use according to the present invention is characterized by a dedicated metering valve system. It is in fact surprisingly found that the use of a dedicated metering valve further increases the apparent pH buffering action of the coated can according to the invention, being also beneficial in terms of residual formoterol, overall stability and efficacy of the formulation. Generally, the can of a pMDI device is crimped with a metering valve for delivering a therapeutically effective dose of the active ingredients. The metering valve assembly comprises at least a gasket seal. Preferably, the valve comprises 2 or 3 gaskets made of the same or different material. In this respect, according to the present invention, the valve is provided with 2 or 3 gaskets, made of the same material or different. Thus, according to the present invention, at least one gasket is made of a proper elastomeric material comprising at least one of polymer selected from: low-density polyethylene, butyl such as chlorobutyl or bromobutyl, butadiene-acrylonitrile, neoprene, EPDM (a polymer of ethylenepropylenediene monomer), TPE (thermoplastic elastomer), cycloolefin copolymer (COC) or combination thereof.

Preferably the valve is provided with 3 gaskets, even more preferably all of them made of EPDM, and herein referred as B-valve.

In one preferred embodiment, the valve is provided with a gasket made of COC, along with two gaskets made of EPDM, and herein referred as A-valve.

In one equally preferred embodiment, the valve is provided with two gaskets, preferably both of them made of chlorobutyl polymer, and herein referred as V-valve.

In one additional preferred embodiment, the valve is provided with a gasket made of butyl rubber, along with two gaskets made of EPDM.

In one additional embodiment, the valve is provided with two gaskets preferably made of bromobutyl, along with one gasket made of a material selected from the group consisting of chlorobutyl, butadiene-acrylonitrile, neoprene, EPDM (a polymer of ethylenepropylenediene monomer), TPE (thermoplastic elastomer), cycloolefin copolymer (COC) or combination thereof. Preferably, the valve is provided with two gaskets made of bromobutyl, along with one gasket made of EPDM.

The metering valve according to the invention is typically capable of delivering a volume in the range from 25 to 150 µl, preferably in the range from 50 to 100 µl, and more preferably of 50 µl or 70 µl per actuation. Suitable valves for the present invention are available on the market, e.g. from manufactures well known in the field.

As further advantage, we have surprisingly found that the choice of the valve may conveniently improve the efficacy and reliability of the final pMDI device. For example, when the HFA152a propellant is used in a coated can according to the present invention, the A-valve or the V-valve provides for an even further improvement of the stability of the final formulation, over e.g. the B-valve which are provided with 3 gaskets made of EPDM.

This improvement in the stability is further enhanced if the formulation is in the form of a solution, as indicated in the present experimental part. The B-valve, in fact, when used in combination with the HFA152a propellant, may lead to a leakage of said propellant, that may result in an undesired loss of product, and possibly compromise the efficacy of the pMDI device over the time. Surprisingly, when the A-valve or the V-valve is used in combination with the HFA152a propellant in a coated can according to the invention, not only the apparent pH buffer action is maximized, but also the leakage of the formulation is substantially avoided. This results in an effective and convenient system to be readily employed in a final pMDI device. This versatility confers a broad use and possibilities of customization of the final pMDI device containing the can according to the invention, thus accomplishing a variety of needs and requirements of the patients and/or of the market.

According to a preferred embodiment, the valve is selected from A-valve and V-valve, being A-valve even more preferred.

Thus, in one preferred embodiment, the invention refers to a FEP coated can for use in a pMDI device, said FEP coated can containing a formulation comprising at least BDP, formoterol fumarate dihydrate, glycopyrronium bromide, HCl and HFA152a propellant, said FEP coated can having a valve selected from A-valve or V-valve. According to this embodiment, the can optionally further comprises ethanol, preferably anhydrous.

The coated can for use in a pMDI device according to the present invention may be filled with the selected formulation by means of common methodologies used in the field. As a general example said methodology may comprise the steps of:
a) preparing a solution comprising: formoterol fumarate, BDP, glycopyrronium bromide and ethanol;
b) filling a FEP coated can with said solution;
c) adding an amount of HCl resulting in a molar ratio between formoterol fumarate dihydrate and the acid comprised between 0.50 to 1.50;
d) adding 1,1-difluoroethane (HFA 152a) propellant;
e) crimping with an Aptar valve and gassing.

The pMDI comprising the coated can according to the invention may have the configuration and components of a commonly used pMDI device, such as those already on the market for well-known formulations for treating e.g. asthma and/or COPD.

Unless otherwise provided, it is intended that all the above embodiments may be combined together and are to be considered as part of the scope of the present invention.

The invention will be now described by the following not limiting examples.

### EXPERIMENTAL PART

In the below Examples 1 and 2, the apparent pH is measured using a standard LiCl electrode commonly used to measure the pH in organic media. Being MDI pressurized product, in order to measure the apparent pH of the formulation the following procedure was applied:
1. Cool down the canister up to at least -50° C. (deeping the canister in a dry ice bath or in liquid nitrogen, to allow to reduce the internal pressure to the atmospheric one).
2. Open the canister by cutting the valve and let the propellant evaporate at room temperature.
3. The remaining ethanolic solution (containing the API) is poured in a glass vial and bring to 10 ml volume with ethanol anhydrous to have a sufficient volume to be measured via a standard LiCl electrode.
4. Measure the apparent pH of the reconstituted solution using an LiCl electrode.

### Example 1

An aluminum FEP coated can according to the invention was filled with a solution comprising FF (0.011 %w/w ), BDP (0.18%w/w), glycopyrronium bromide (0.022% w/w), HCl 1 M ( w/w) and Ethanol (12%w/w), in the presence of HFA152a.

The aluminum FEP coated can filled with the above solution and provided with valves A, B or V were put in stability chambers at 25C°, 60% R.H. (relative humidity).

The Apparent pH (App pH) and the residual percentage of formoterol fumarate dihydrate (FF% w/w), over the initial content (100% at T=0) of the solution were measured at T=0, after 1, 3 and 6 months respectively.

Results are collected in Table 1 below.

Table 1

Apparent pH value (App pH) and FF% in FEP coated can at T=0 and T=1 month (1M); T=3 months (3 M) and 6 months (6 M), measured at 25° C./60% R.H..

| Propellant | Can | Valve | T=0 FF% (App pH) | T=1 M FF% (App pH) | T=3 M FF% (App pH) | T=6 M FF% (App pH) |
|---|---|---|---|---|---|---|
| 152a | FEP | A-valve | 100.0 (4.5) | 98.9 (4.5) | 96.9 (4.5) | 97.7 (4.4) |
| 152a | FEP | V-valve | 100.0 (4.4) | 98.0 (4.4) | 95.5 (4.3) | 94.4 (4.3) |
| 152a | FEP | B-valve | 100 (4.5) | 99.1 (4.4) | 97.5 (4.5) | 96.7 (4.3) |

A-valve: a valve provided with a gasket made of COC, along with two gaskets made of EPDM, as e.g. available by Aptar.
V-valve: a valve provided with two gaskets, both of them made of chlorobutyl polymer, as e.g. available by Vari.
B-valve: a valve provided with 3 gaskets, all of them made of EPDM, as e.g. available by Bespak.

### Example 2 (Comparative)

The same analysis of Example 1 has been ran using uncoated aluminum can provided with valves A, B or V.

The Apparent pH (App pH) of solution according to Example 1 were measured at T=0, after 1, 3 and 6 months respectively. Results are collected in Table 2.

Table 2

apparent pH value (App pH) and FF% w/w in uncoated can at T=0 and T=1 month (1 M); T=3 months (3 M) and 6 months (6 M), measured at 25° C./60% R.H..

| Propellant | Uncoated CAN | Valve | T=0 (App pH) | T=1M (App pH) | T=3M (App pH) | T=6M (App pH) |
|---|---|---|---|---|---|---|
| 152a | Al | B-valve | (4.5) | (4.9) | (5.3) | (5.3) |
| 152a | Al | A-valve | (4.5) | (5.1) | (5.6) | (5.7) |
| 152a | Al | V-valve | (4.7) | (5.0) | (5.6) | (5.5) |

B-valve: a valve provided with 3 gaskets, all of them made of EPDM, as e.g. available by Bespak.
A-valve: a valve provided with a gasket made of COC, along with two gaskets made of EPDM, as e.g. available by Aptar.
V-valve: a valve provided with two gaskets, both of them made of chlorobutyl polymer, as e.g. available by Vari.

As evident from the above Tables 1 and 2 the use of a FEP coated can filled with a solution in presence of HFA152a propellant according to the invention provided with the indicated Valves, guarantees a convenient stabilization of the pH of the therein contained solution, even for prolonged period of time, e.g. even after 6 months, when compared to T=0.

On the contrary, by using an uncoated can (comparative), the pH substantially increases with respect to the measure at T=0, also leading to a potential decreasing of the FF% w/w, even after just one month of storage at 25° C., which can be assumed to be the room temperature.

1. A can for use in a pMDI device, said can containing a formulation comprising at least a corticosteroid, a LABA agent, a LAMA agent and a HFA 152a or HFO propellant, being said can internally coated by a coating comprising at least a compound selected from an epoxy-phenol resin, a perfluorinated polymer, a perfluoroalkoxyalkane polymer, a perfluoroalkoxyalkylene polymer, a perfluoroalkylene polymer, poly-tetrafluoroethylene polymer (Teflon), fluorinated-ethylene-propylene polymer (FEP), polyether sulfone polymer (PES), a fluorinated-ethylene-propylene polyether sulfone polymer (FEP-PES), a polyamide, polyimide, polyamideimide, polyphenylene sulfide, plasma, mixtures or combinations thereof, wherein said can is provided with a valve having at least one gasket made of a material comprising at least one polymer selected from low-density polyethylene, butyl such as chlorobutyl or bromobutyl, butadiene-acrylonitrile, neoprene, EPDM (a polymer of ethylenepropylenediene monomer), TPE (thermoplastic elastomer), cycloolefin copolymer (COC) or combination thereof.

2. The can according to claim 1, wherein said corticosteroid is selected from the group consisting of: budesonide, beclomethasone dipropionate, flunisolide, fluticasone, ciclesonide, mometasone, mometasone desonide, rofleponide, hydrocortisone, prednisone, prednisolone, methyl prednisolone, naflocort, deflazacort, halopredone acetate, fluocinolone acetonide, fluocinonide, clocortolone, tipredane, prednicarbate, alclometasone dipropionate, halometasone, rimexolone, deprodone propionate, triamcinolone, betamethasone, fludrocoritisone, desoxycorticosterone, rofleponide and etiprednol dicloacetate.

3. The can according to claim 2, wherein said corticosteroid is beclomethasone dipropionate or budesonide.

4. The can according to any one of the preceding claims, wherein the LABA agent is selected from the group consisting of: fenoterol, formoterol fumarate, formoterol fumarate dihydrate, arformoterol, carmoterol, indacaterol, milveterol, bambuterol, clenbuterol, vilanterol, olodaterol, abediterol, terbultaline and salmeterol.

5. The can according to claim 4, wherein said LABA agent is formoterol fumarate dihydrate.

6. The can according to claims 1, wherein the formulation agent alternatively comprises an agent selected from the group consisting of salbutamol and (R)-salbutamol.

7. The can according to one of the preceding claims wherein the LAMA agent is selected from the group consisting of glycopyrronium, methscopolamine, ipratropium, oxitropium, trospium, tiotropium, aclidinium and umeclidinium or pharmaceutically acceptable salts.

8. The can according to claim 7, wherein said LAMA agent is glycopyrronium bromide.

9. The can according to any one of the preceding claims, wherein the HFO propellant is selected from the group consisting of: 1,3,3,3-tetrafluoropropene (HFO-1234ze) and 2,3,3,3-tetrafluoropropene (HFO-1234yf).

10. The can according to any one of the preceding claims, internally coated by a coating comprising a fluorinated-ethylene-propylene (FEP) polymer.

11. The can according to any one of the preceding claims, containing a formulation further comprising one or more excipients, co-solvents and acids.

12. The can according to claim 11, wherein said co-solvent is an aliphatic alcohol having from 1 to 4 carbon atoms.

13. The can according to claim 12, wherein said aliphatic alcohol is ethanol, preferably anhydrous.

14. The can according to claims 11-13, containing a formulation further comprising a mineral or organic acid selected from the group consisting of: hydrochloric, hydrobromic, nitric, fumaric, phosphoric and citric acid, maleic acid, acetic acid, xinafoic acid, oxalic acid, lactic acid, 2-methyl propionic acid, malic acid, butanoic acid, tartaric acid, propionic acid, pentanoic acid, succinic acid, glycolic acid, hexanoic acid, malonic acid, glutaric acid, formic ac-id, adipic acid, ascorbic acid, benzoic acid and glucuronic acid.

15. The can according to claim 14, wherein said acid is hydrochloric acid.

16. The can according to any one of the preceding claims, containing a formulation further comprising a low volatility component selected from the group consisting of: glycols, propylene glycol, polyethylene glycol, glycerol or esters thereof, ascorbyl palmitate, isopropyl myristate.

17. The can according to any one of the preceding claims, containing a formulation in form of a solution.

18. The can according to claims 1 to 17, wherein the valve is provided with 3 gaskets all of them made of EPDM.

19. The can according to claims 1 to 17, wherein the valve is provided with a gasket made of COC, along with two gaskets made of EPDM.

20. The can according to claims 1 to 17 wherein the valve is provided with two gaskets, both of them made of chlorobutyl polymer.

21. The can according to claims 1 to 17, wherein the valve is provided with a gasket made of butyl rubber, along with two gaskets made of EPDM.

22. The can according to claims 1 to 17, wherein the valve is provided with two gaskets made of bromobutyl, along with one gasket made of a material selected from the group consisting of chlorobutyl, butadiene-acrylonitrile, neoprene, EPDM (a polymer of ethylenepropylenediene monomer), TPE (thermoplastic elastomer), cycloolefin copolymer (COC) or combination thereof.

23. The can according to any one of claims 1 to 22, wherein the propellant is HFA152a and the valve is provided with a gasket made of COC, along with two gaskets made of EPDM; or the valve is provided with two gaskets, both of them made of chlorobutyl polymer.

24. The can according to any one of the preceding claims, containing a formulation having an apparent pH buffered between 2.5 and 5.

25. The can according to claim 24, containing a formulation having an apparent pH buffered between 3 and 4.5.

26. A pMDI device comprising the can according to any one of the preceding claims.

27. The pMDI device according to claim 26 for the treatment of a respiratory disease selected from asthma and/or COPD.
